(19) United States
(12) Patent Application Publication
(10) Pub. No.: US 2023/0310886 A1
(43) Pub. Date: Oct. 5, 2023

(54) BRACHYTHERAPY TREATMENT SYSTEM AND METHOD OF OPERATING THE SYSTEM

(71) Applicant: Varian Medical Systems, Inc., Palo Alto, CA (US)

(72) Inventors: Rebecca PARK, West Sussex (GB); Sophie WETHERALL, Worcestershire (GB); Elena NIOUTSIKOU, Erlangen (DE)

(21) Appl. No.: 17/710,970

(22) Filed: Mar. 31, 2022

(51) Int. Cl. A61N 5/10

(52) U.S. Cl. CPC A61N 5/1027; A61N 5/1007; A61N 5/103; A61N 5/1049; A61N 5/1014

(57) ABSTRACT

Misconnected applicators are eliminated in a brachytherapy treatment system by associating the output channels of an afterloader with the channel numbers after the applicators have been coupled to the output channels. In addition, the brachytherapy treatment system ensures the delivery of a proper dose by identifying the exact locations of the distal ends of the applicators right before the radiation treatment is to begin, and determining the final dose based on the exact locations.

### BACKGROUND OF THE INVENTION

### 1. Field of the Invention

The present invention relates to brachytherapy and, more particularly, to a brachytherapy treatment system and a method of operating the system.

### 2. Description of the Related Art

Brachytherapy is a method of treating a cancerous tumor with radiation. FIG. 1 shows a block diagram that illustrates a prior-art brachytherapy treatment system 100. As shown in FIG. 1, system 100 includes an applicator 110 that has a proximate end and a distal end, a guide tube 112 coupled to the proximate end of applicator 110, and an afterloader 116 that has an output channel 118 coupled to guide tube 112.

To provide a radiation treatment, applicator 110 is inserted inside a body such that the distal end of applicator 110 is placed close to the location of a tumor. Following the insertion of applicator 110, afterloader 116 uses controllers and drive mechanisms to advance a radioactive source from output channel 118 through guide tube 112 to the distal end of applicator 110 and then, after a preplanned treatment period of time, retract the radioactive source from applicator 110 back through guide tube 112 and into afterloader 116 where the radioactive source is safely held.

Afterloaders commonly have one radioactive source and multiple output channels down which the radioactive source can travel. The multiple output channels can be coupled to multiple guide tubes which, in turn, are coupled to multiple applicators that are positioned around the tumor to allow the tumor to be sequentially radiated from different directions with the radioactive source.

In the field of high dose rate (HDR) and pulsed dose rate (PDR) brachytherapy, very high doses of radiation with steep gradients are delivered within seconds in order to treat solid cancers. Since the dose drops off steeply, shifts in the treatment position versus a planned dose position can make a big difference in the effectivity and complication rates.

One reason doses can be incorrectly delivered when multiple channels are used is that an operator can accidentally connect the applicators, via the guide tubes, to the wrong output channels on the afterloader. When a treatment plan is established, different applicators are assigned different source positions and dwell times and given a channel number that this part of the plan will deliver.

Incorrectly coupling the applicators to the afterloader can result in the wrong part of the plan being applied to the wrong applicator, thereby changing the overall dose distribution of the plan. To address this issue, a second operator is frequently assigned to cross check and verify that the applicators, via the guide tubes, have been correctly coupled to the output channels on the afterloader and that they match what was planned.

Although a second operator substantially reduces the likelihood that the guide tubes are incorrectly connected, accidents still occur. In addition, the use of a second operator is both time consuming and labor intensive, which drives up costs. Further, if an incorrectly connected guide tube and applicator are detected (which usually happens late in the process), correcting for the error adds even more time to a procedure, where brevity matters (patients are sedated or anesthetized).

Other reasons that doses are incorrectly delivered include incorrectly identifying the location of the distal end of the applicator, organ filling/swelling between the time of treatment planning and treatment delivery, shifting of one or more applicators due to patient movement, and wrongly identifying catheters.

Thus, there is a need for a brachytherapy treatment system that eliminates misconnecting applicators to output channels, and delivers an accurate radiation dose even when organ filling/swelling and applicator movement has occurred.

### SUMMARY OF THE INVENTION

The brachytherapy treatment system of the present invention eliminates misconnecting applicators to output channels, and delivers an accurate radiation dose even when organ filling/swelling and applicator movement has occurred. The brachytherapy treatment system includes an applicator that delivers radiation to a tumor. The applicator has a proximate end and a distal end. The system also includes a guide tube that is coupled to the proximate end of the applicator, and an imager that takes an anatomical image and a focused image. The system additionally includes an afterloader that is coupled to the guide tube and the imager. The afterloader drives a dummy device from an output channel of the afterloader to the distal end of the applicator, commands the imager to take the focused image after the dummy device has reached the distal end of the applicator, and retracts the dummy device after the focused image has been taken. The dummy device has a high contrast to the imager. The system further includes a treatment planning system coupled to the imager and the afterloader. The treatment planning system receives the anatomical and focused images, and determines a treatment time for a radioactive source for the output channel and the applicator based on the anatomical and focused images.

The present invention also includes a method of operating a brachytherapy treatment system. The method includes taking an anatomical image with an imager of a patient to determine shapes and locations of a tumor and any organs at risk. The method also includes driving a dummy device from an output channel of an afterloader to a distal end of an applicator. The dummy device has a high contrast to the imager. The method additionally includes taking a focused image with the imager of the dummy device inside the whole length of the applicator, all the way to its distal end, and retracting the dummy device into the afterloader after the focused image has been taken. The method further includes determining an exact location of the distal end of the applicator based on one or both the anatomical image and the focused image, and determining a treatment time for a radiation source for the applicator based on the location of the tumor, any organs at risk, and the exact location of the distal end of the applicator.

The present invention includes a second method of operating a brachytherapy treatment system. The method includes taking an anatomical image with an imager of a patient to determine shapes and locations of a tumor and any organs at risk. The method also includes sequentially driving a dummy device from a plurality of output channels of an afterloader to the distal ends of a plurality applicators. The dummy device has a high contrast to an imager. The method also includes taking a plurality of focused images with the imager of the dummy device such that a focused image is taken of the dummy device driven all the way to the distal end of each applicator. The method additionally includes retracting the dummy device into the afterloader after each focused image has been taken, and determining the exact locations of the distal ends of the applicators based on one or both the anatomical image and the focused images. The method further includes determining a treatment time for a radiation source for the applicator associated with each output channel based on the location of the tumor, any organs at risk, and the exact locations of the applicators.

A better understanding of the features and advantages of the present invention will be obtained by reference to the following detailed description and accompanying drawings which set forth an illustrative embodiment in which the principles of the invention are utilized.

### BRIEF DESCRIPTION OF THE DRAWINGS

The accompanying drawings described herein are used for providing further understanding of the present application and constitute a part of the present application. Exemplary embodiments of the present application and the description thereof are used for explaining the present application and do not constitute limitations on the present application.

FIG. 1 is a block diagram illustrating a prior-art brachytherapy treatment system 100.

FIG. 2 is a block diagram illustrating an example of a brachytherapy treatment system 200 in accordance with the present invention.

FIGS. 3A-3B are a flow chart illustrating an example of a method 300 of operating a multi-channel brachytherapy treatment system in accordance with the present invention.

FIGS. 4A-4B are a flow chart illustrating an example of an alternate method 400 of operating a multi-channel brachytherapy treatment system in accordance with the present invention.

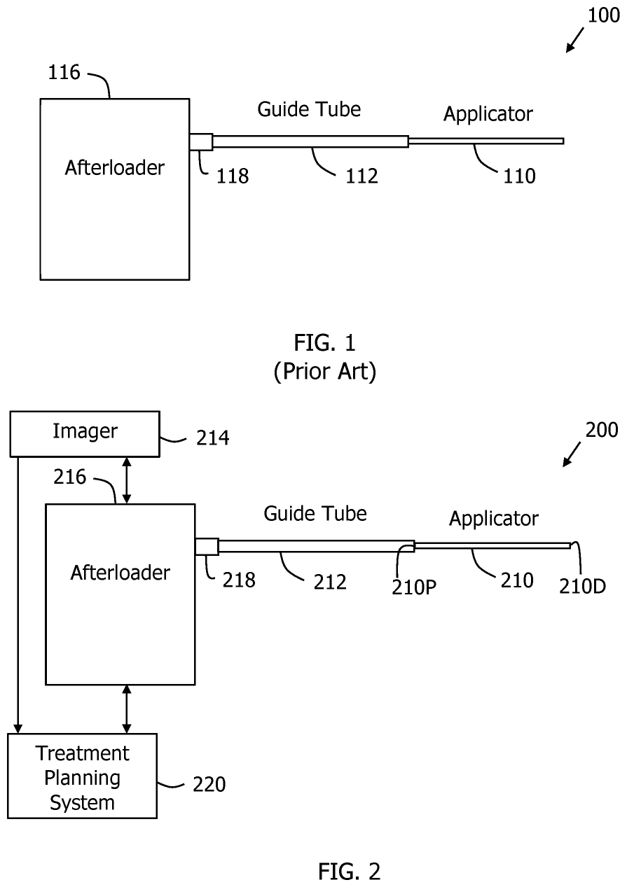

### DETAILED DESCRIPTION OF THE INVENTION

FIG. 2 shows a block diagram that illustrates an example of a brachytherapy treatment system 200 in accordance with the present invention. As described in greater detail below, system 200 eliminates misconnected applicators by associating the output channels of an afterloader with the applicators after the applicators have been coupled to the output channels, and ensures the delivery of a proper dose by determining the exact locations of the distal ends of the applicators right before radiation is to begin, and then determining the final dose based on the exact locations.

As shown in FIG. 2, brachytherapy treatment system 200 includes an applicator 210 that delivers radiation to a tumor. Applicator 210, which has a proximate end 210P and a distal end 210D, can be implemented in a number of ways, such as with an interstitial needle, an intracavity device, or a catheter. System 200 also includes a guide tube 212 that is coupled to the proximate end 210P of applicator 210.

In addition, system 200 includes an imager 214 that takes an anatomical image of a region of a body and a focused image of applicator 210 positioned in the body from the same frame of reference. The anatomical image includes an image of a tumor and images of any organs at risk. In one embodiment, the anatomical image also includes an image of applicator 210, including the proximate and distal ends 210P and 210D. In another embodiment, the anatomical image does not include an image of applicator 210, and yet in a further embodiment, a digital representation of applicator 210 is added to the anatomical image data from the original anatomical image.

The focused image includes the distal end 210D of applicator 210. In one embodiment, the focused image does not include the proximate end 210P, while in another embodiment the focused image includes both ends of applicator 210, but does not include an image of the tumor. When the anatomical image includes an image of applicator 210, the focused image need only be of the distal end 210D of applicator 210 since the images are taken from the same frame of reference.

Similarly, when the focused image includes both ends of applicator 210, the anatomical image does not need to include an image of applicator 210 or a digital representation since the anatomical and focused images are taken from the same frame of reference. The focused image can represent one image, or the summation of a number of images. Imager 214 can be implemented in a conventional manner, such as with a computerized tomography (CT) or magnetic resonance (MR) imager, or a PET/CT imager, or a SPECT/CT imager or a C-arm imager.

As further shown in FIG. 2, brachytherapy treatment system 200 includes an afterloader 216 that has an output channel 218 coupled to guide tube 212. Prior to providing radiation during a treatment session, afterloader 216 uses conventional controllers and mechanisms to drive a dummy device from output channel 218 through guide tube 212 to the distal end 210D of applicator 210. The dummy device has a high contrast to imager 214.

Afterloader 216 detects when the dummy device has reached the distal end 210D of applicator 210 and, in response, commands imager 214 to take the focused image. Imager 214 takes the image in response to the command, and notifies afterloader 216 once the focused image has been taken. In response to the notification, afterloader 216 retracts the dummy device back into afterloader 216.

Brachytherapy treatment system 200 also includes a treatment planning system 220 that is coupled to imager 214 and afterloader 216. Treatment planning system 220 receives the anatomical and focused images, determines the locations of the tumor and any organs at risk from the anatomical image using conventional methods, determines the location of the dummy device from the focused image using conventional methods to determine the exact location of the distal end 210D of applicator 210, and determines a final treatment plan that identifies a radioactive source and a treatment time for output channel 218 and applicator 210 based on the locations of the tumor, any organs at risk, and the distal end 210D of applicator 210.

One advantage of the present invention is that the dummy device allows treatment planning system 220 to accurately determine the location of the distal end 210D of applicator 210 immediately before the radiation treatment is performed. Any unintended movement of the distal end 210D of applicator 210, such as from movement of the patient or organ filling/swelling, can lead to the wrong dose being applied.

Thus, by determining the exact location of the distal end 210D of applicator 210 right before radiation is applied, any unintended movement of the distal end 210D of applicator 210 can be identified before treatment begins. When any unintended movement is detected, the proper dose can be determined for the final treatment plan based on the current position of the distal end 210D of applicator 210.

After the treatment plan has been finalized with the exact location of the distal end 210D of applicator 210, and then approved, afterloader 216 drives the radioactive source from output channel 218 through guide tube 212 to the distal end 210D of applicator 210, starts the treatment time, and retracts the radioactive source back into afterloader 216 where it is safely held after the treatment time has expired, thereby completing the radiation treatment.

Brachytherapy treatment system 200 is illustrated with a single applicator 210, a single guide tube 212, and a single channel 218 for simplicity. Alternately, system 200 can include multiple applicators, multiple guide tubes that are coupled to the applicators, and an afterloader with multiple channels that are coupled to the guide tubes. The multiple channels are identified by channel numbers.

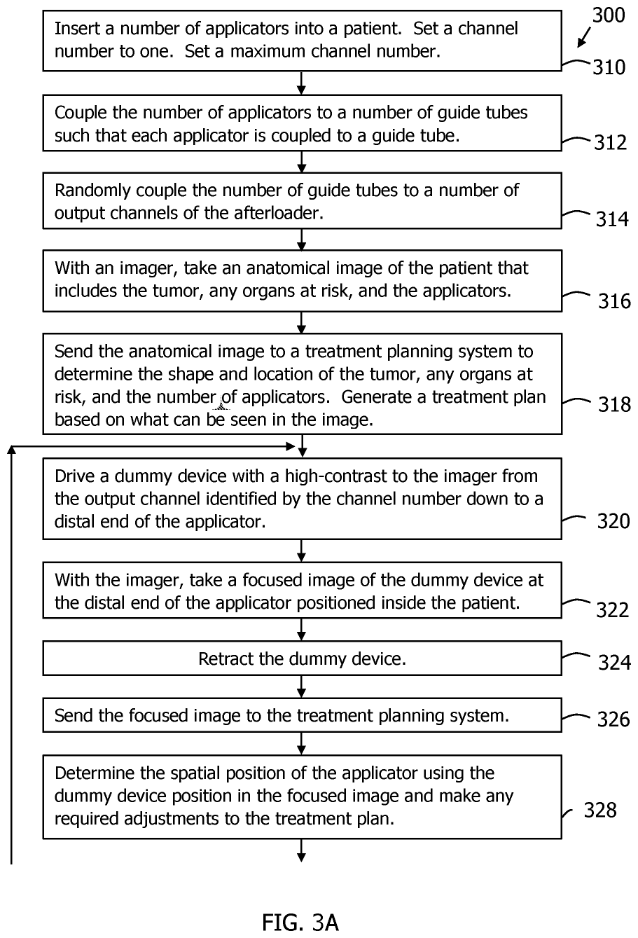

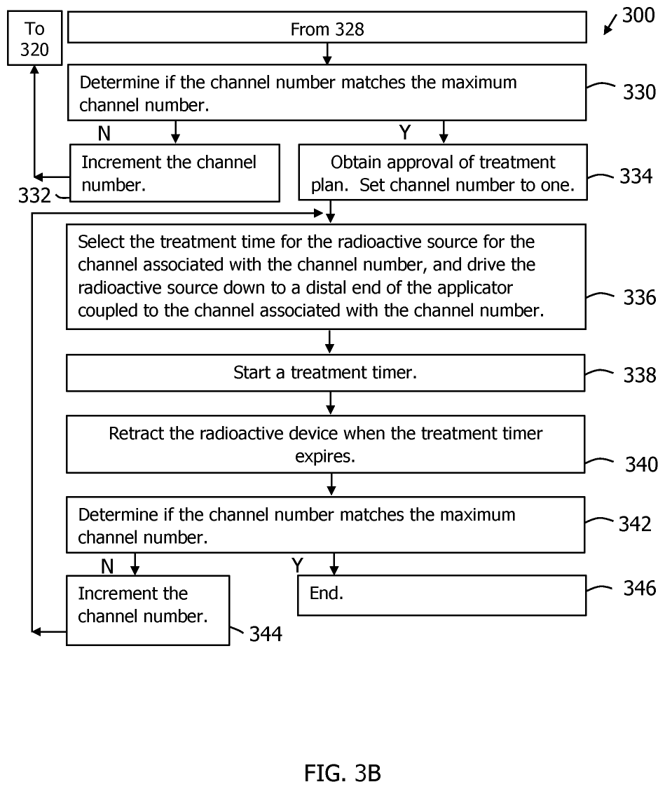

FIGS. 3A-3B show a flow chart that illustrates an example of a method 300 of operating a multi-channel brachytherapy treatment system in accordance with the present invention. As shown in FIGS. 3A-3B, method 300 begins at 310 by inserting a number of applicators into a patient so that the distal ends of the applicators are positioned close to a tumor. In addition, a channel number is set to one, and a maximum channel number is set. The channel number identifies one of the output channels of an afterloader, such as afterloader 216.

After the applicators have been inserted into the patient, method 300 moves to 312 to couple the number of applicators to a number of guide tubes so that each applicator is coupled to a guide tube. Following this, method 300 moves to 314 to randomly couple the number of applicators, via the guide tubes, to the number of output channels of the afterloader so that each applicator is coupled to an output channel. 310-314 can be performed in a different order.

Method 300 next moves to 316 to take an anatomical image of the patient that includes the tumor, any organs at risk, and the applicators. In the present embodiment, afterloader 216 outputs a capture-image message to imager 214 to take the anatomical image in response to an operator-initiated command.

Once the anatomical image has been captured, method 300 moves to 318 to send the anatomical image to a treatment planning system, such as treatment planning system 220, to determine the shape and location of the tumor, the shape and location of any organs at risk, and the shape and location of the applicators. Conventional methods can be used to determine the shapes and locations of the targets and the organs at risk from the anatomical image data. In the present embodiment, imager 214 sends the anatomical image to treatment planning system 220 as anatomical image data.

In addition, treatment planning system 220 generates a treatment plan based on what can be seen in the image. The treatment plan includes determining a treatment time for the radioactive source for the channel number using conventional methods based on the current position of the distal end of the applicator, the tumor, and any organs at risk.

After this, method 300 moves to 320 to drive a dummy device, which has a high contrast to imager 214, such as a wire, from the output channel of the afterloader through the guide tube to the distal end of the applicator that is coupled to the output channel identified by the channel number.

Next, method 300 moves to 322 to take a focused image of the high-contrast dummy device at the distal end of the applicator. The focused image represents an image of the distal end of the applicator. In the present embodiment, afterloader 216 drives the dummy device to the end of the applicator, sends a capture-image message to imager 214 to take the focused image after the dummy device has reached the distal end 210D of applicator 210, and retracts the dummy device after the focused image has been taken. Imager 214 notifies afterloader 216 when the focused image has been taken, and sends the focused image to treatment planning system 220 as focused image data.

Method 300 next moves to 328 where the treatment planning system determines the spatial position of the applicator using the position of the dummy device in the focused image and make any required adjustments to the treatment plan.

Following this, method 300 moves to 330 to determine if the channel number is equal to the maximum channel number. When the numbers are unequal, method 300 moves to 332 to increment the channel number, and then returns to 320 to process a next channel number.

When the numbers are equal, which indicates that the exact locations of the distal ends of all of the applicators have been determined, method 300 moves to 334 to obtain approval of the treatment plan and again set the channel number to one. Following this, method 300 moves to 336 to select the treatment time for the radioactive source for the output channel identified by the channel number, and drive the radioactive source from the output channel through the guide tube to the distal end of the applicator coupled to the channel identified by the channel number.

Next, method 300 moves to 338 to start a treatment timer, and then moves to 340 to retract the radioactive source back into afterloader 216 where it is safely held after the treatment time has expired. Method 300 next moves to 342 to determine if the channel number is equal to the maximum channel number. When the numbers are unequal, method 300 moves to 344 to increment the channel number, and then returns to 336 to process a radiation treatment for the next channel number. When the numbers are equal, method 300 moves to 346 to end.

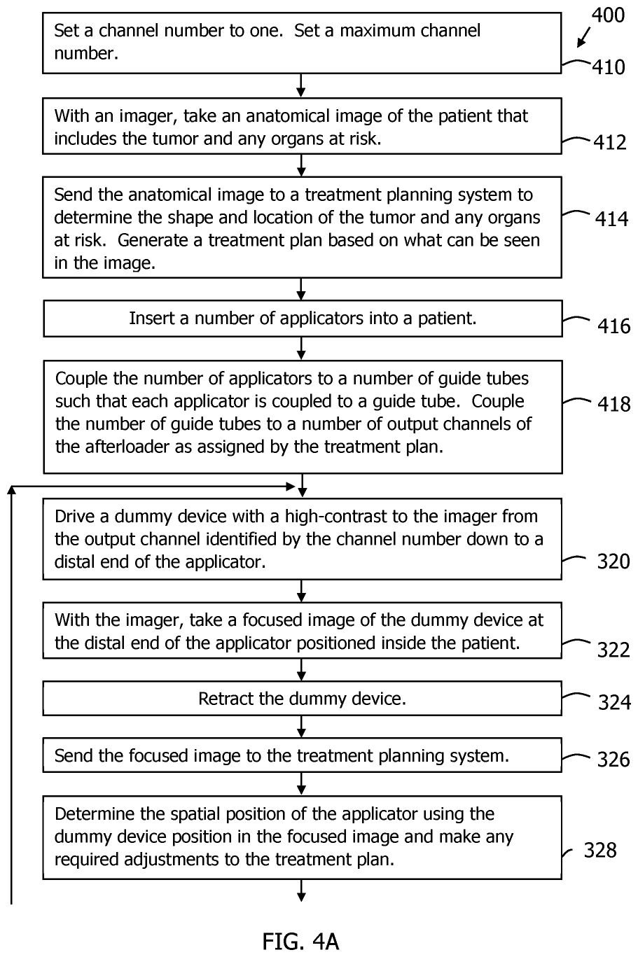

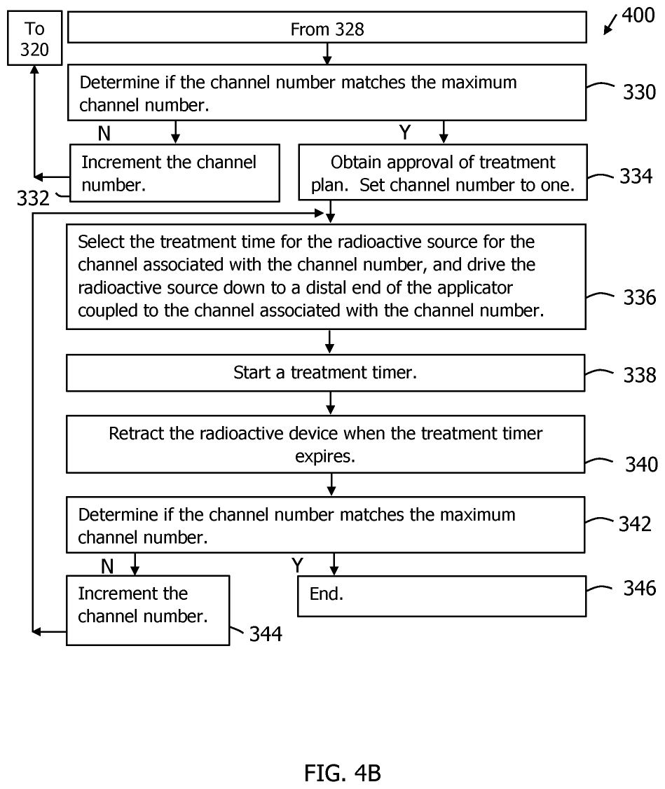

FIGS. 4A-4B show a flow chart that illustrates an example of an alternate method 400 of operating a multi-channel brachytherapy treatment system in accordance with the present invention. Method 400 is similar to method 300 and, as a result, utilizes the same reference numerals to designate the steps that are common to both methods.

As shown in FIG. 4, method 400 begins at 410 by setting a channel number to one and setting a maximum channel number. 410 differs from 310 in that 410 does not insert the applicators at this time. Next, method 400 moves to 412 to take an anatomical image of the patient that includes the tumor and any organs at risk.

Following this, method 400 moves to 414 to send the anatomical image to a treatment planning system, such as treatment planning system 220, to determine the shape and location of the tumor and the shape and location of any organs at risk. Conventional methods can be used to determine the shapes and locations from the anatomical image data. In the present embodiment, imager 214 sends the anatomical image to treatment planning system 220 as anatomical image data. In addition, treatment planning system 220 generates a treatment plan based on what can be seen in the image, which no longer includes an image of the applicators.

In a first embodiment, a digital representation of the applicators is inserted into the anatomical image data derived from the anatomical image. The treatment plan includes determining a treatment time for the radioactive source for the channel number using conventional methods based on the positions ( ) of the digital representations of the applicators, the tumor, and any organs at risk. In a second embodiment, anatomical image data from the anatomical image is left without a digital representation of the image of the applicators.

In some embodiments, the anatomical and focused images are taken during the same treatment session that radiation is provided. In other embodiments, the anatomical image can be taken and the treatment plan can be prepared a significant time before the focused images are taken. When this happens, applicators are not inserted into the patient before taking the anatomical image, but are inserted prior to taking the focused images.

After this, method 400 moves to 416 to insert a number of applicators into a patient. Method 400 then moves to 418 to couple the number of applicators to a number of guide tubes such that each applicator is coupled to a guide tube. Method 400 also couples the number of guide tubes to a number of output channels of the afterloader as assigned by the treatment plan. 416 and 418 can be performed in a different order.

After this, method 400 continues on in the same manner as method 300, next moving to 320 to drive a dummy device, and then to 322 to take a focused image. In methods 300 and 400, the focused image represents an image of the distal end of the applicator wherein the image does not include an image of the proximate end of the applicator.

Alternately, the focused image can represent multiple images that include both ends of the applicator while the multiple images do not include an image of the tumor. Additional focused images can be taken to fully identify the whole length of the applicator. Fully identifying the whole length of an applicator eliminates the need for the anatomical image to resolve the applicators accurately or a digital representation of the applicators since the whole applicator is detected in the image space. Once the focused image has been captured, method 400 moves to 324 to retract the dummy device, and then continues on as before in method 300.

Another advantage of the present invention is that, regardless of whether the applicators are randomly coupled to the output channels or coupled to the output channels based on the treatment plan, the association of the output channels to the channel numbers occurs after the applicators have been coupled to the channels. This eliminates the possibility that the applicators can be coupled to the wrong output channels of the afterloader, thereby eliminating the need for a second operator.

Further, when a significant period of time separates the anatomical and focused images, a second anatomical image can optionally be taken before taking the focused image to detect any organ filling/swelling that has occurred, which also shows the in-patient locations of the actual applicators.

Reference has now been made in detail to the various embodiments of the present disclosure, examples of which are illustrated in the accompanying drawings. While described in conjunction with the various embodiments, it will be understood that these various embodiments are not intended to limit the present disclosure. On the contrary, the present disclosure is intended to cover alternatives, modifications and equivalents, which may be included within the scope of the present disclosure as construed according to the claims.

Furthermore, in the preceding detailed description of various embodiments of the present disclosure, numerous specific details are set forth in order to provide a thorough understanding of the present disclosure. However, it will be recognized by one of ordinary skill in the art that the present disclosure may be practiced without these specific details or with equivalents thereof. In other instances, well-known methods, procedures, components, and circuits have not been described in detail so as not to unnecessarily obscure aspects of various embodiments of the present disclosure.

It is noted that although a method may be depicted herein as a sequence of operations for clarity, the described sequence of operations does not necessarily dictate the order of the operations. It should be understood that some of the operations may be skipped, performed in parallel, or performed without the requirement of maintaining a strict order of sequence.

The drawings showing various embodiments in accordance with the present disclosure are semi-diagrammatic and not to scale and, particularly, some of the dimensions are for the clarity of presentation and are shown exaggerated in the drawing Figures. Similarly, although the views in the drawings for the ease of description generally show similar orientations, this depiction in the Figures is arbitrary for the most part. Generally, the various embodiments in accordance with the present disclosure can be operated in any orientation.

Some portions of the detailed descriptions may be presented in terms of procedures, logic blocks, processing, and other symbolic representations of operations on data bits within a computer memory. These descriptions and representations are used by those skilled in the data processing arts to effectively convey the substance of their work to others skilled in the art.

In the present disclosure, a procedure, logic block, process, or the like, is conceived to be a self-consistent sequence of operations or instructions leading to a desired result. The operations are those utilizing physical manipulations of physical quantities. Usually, although not necessarily, these quantities take the form of electrical or magnetic signals capable of being stored, transferred, combined, compared, and otherwise manipulated in a computing system. It has proven convenient at times, principally for reasons of common usage, to refer to these signals as transactions, bits, values, elements, symbols, characters, samples, pixels, or the like.

It should be borne in mind, however, that all of these and similar terms are to be associated with the appropriate physical quantities and are merely convenient labels applied to these quantities. Unless specifically stated otherwise as apparent from the following discussions, it is appreciated that throughout the present disclosure, discussions utilizing terms such as “generating,” “determining,” “assigning,” “aggregating,” “utilizing,” “virtualizing,” “processing,” “accessing,” “executing,” “storing,” or the like, refer to the action and processes of a computer system, or similar electronic computing device or processor.

The processing system, or similar electronic computing device or processor manipulates and transforms data represented as physical (electronic) quantities within the computer system memories, registers, other such information storage, and/or other computer readable media into other data similarly represented as physical quantities within the computer system memories or registers or other such information storage, transmission or display devices.

The technical solutions in the embodiments of the present application have been clearly and completely described in the prior sections with reference to the drawings of the embodiments of the present application. It should be noted that the terms “first,” “second,” and the like in the description and claims of the present invention and in the above drawings are used to distinguish similar objects and are not necessarily used to describe a specific sequence or order. It should be understood that these numbers may be interchanged where appropriate so that the embodiments of the present invention described herein can be implemented in orders other than those illustrated or described herein.

The functions described in the present embodiment, if implemented in the form of a software functional unit and sold or used as a standalone product, can be stored in a computing device readable storage medium. Based on such understanding, a portion of the embodiments of the present application that contributes to the prior art or a portion of the technical solution may be embodied in the form of a software product stored in a storage medium, including a plurality of instructions for causing a computing device (which may be a personal computer, a server, a mobile computing device, or a network device, and so on) to perform all or part of the steps of the methods described in various embodiments of the present application. The foregoing storage medium includes: a USB drive, a portable hard disk, a read-only memory (ROM), a random-access memory (RAM), a magnetic disk, an optical disk, and the like, which can store program code.

The various embodiments in the specification of the present application are described in a progressive manner, and each embodiment focuses on its difference from other embodiments, and the same or similar parts between the various embodiments may be referred to another case. The described embodiments are only a part of the embodiments, rather than all of the embodiments of the present application. All other embodiments obtained by a person of ordinary skill in the art based on the embodiments of the present application without departing from the inventive skills are within the scope of the present application.

The above embodiments are merely used for illustrating rather than limiting the technical solutions of the present invention. Although the present application is described in detail with reference to the foregoing embodiments, those of ordinary skill in the art should understand that the technical solutions recorded in the foregoing embodiments may still be modified or equivalent replacement may be made on part or all of the technical features therein. These modifications or replacements will not make the essence of the corresponding technical solutions be departed from the scope of the technical solutions in the embodiments of the present invention.

It should be understood that the above descriptions are examples of the present invention, and that various alternatives of the invention described herein may be employed in practicing the invention. For example, the various modules can be implemented as cards. Thus, it is intended that the following claims define the scope of the invention and that structures and methods within the scope of these claims and their equivalents be covered thereby.

What is claimed is:

1. A brachytherapy treatment system comprising:
an applicator to deliver radiation to a tumor, the applicator having a proximate end and a distal end;
a guide tube coupled to the proximate end of the applicator;
an imager to take an anatomical image and a focused image;
an afterloader coupled to the guide tube and the imager, the afterloader to drive a dummy device from an output channel of the afterloader to the distal end of the applicator, command the imager to take the focused image after the dummy device has reached the distal end of the applicator, and retract the dummy device after the focused image has been taken, the dummy device having a high contrast to the imager; and
a treatment planning system coupled to the imager and the afterloader, the treatment planning system to receive the anatomical and focused images, and determine a treatment time for a radioactive source for the output channel and the applicator based on the anatomical and focused images.

2. The brachytherapy treatment system of claim 1, wherein the anatomical image includes an image of the tumor and images of any organs at risk.

3. The brachytherapy treatment system of claim 2, wherein anatomical image data from the anatomical image includes one of: image data of the applicator, no image data of the applicator, or a digital representation of the applicator.

4. The brachytherapy treatment system of claim 2, wherein the treatment planning system determines a position of the tumor and positions of any organs at risk from the anatomical image.

5. The brachytherapy treatment system of claim 4, wherein the treatment planning system determines an exact location of the distal end of the applicator from one or both the anatomical and focused images.

6. The brachytherapy treatment system of claim 5, wherein:
the focused image includes the distal end of the applicator; and
the focused image includes one of: the proximate end of the applicator is missing from the focused image, or the proximate end of the applicator is included and an image of the tumor is missing.

7. The brachytherapy treatment system of claim 5, wherein the anatomical and focused images have a same frame of reference.

8. The brachytherapy treatment system of claim 5, wherein the treatment planning system determines a treatment time for a radioactive source based on the exact location of the distal end of the applicator with respect to the position of the tumor and the positions of any organs at risk.

9. The brachytherapy treatment system of claim 8, wherein the afterloader drives the radioactive source from the output channel of the afterloader to the distal end of the applicator.

10. The brachytherapy treatment system of claim 9, wherein:
the afterloader starts the treatment time after the radioactive source has reached the distal end of the applicator; and
the afterloader retracts the radioactive source after the treatment time has expired.

11. A method of operating a brachytherapy treatment system, the method comprising:
taking an anatomical image with an imager of a patient to determine shapes and locations of a tumor and any organs at risk;
driving a dummy device from an output channel of an afterloader to a distal end of an applicator, the dummy device having a high contrast to the imager;
taking a focused image with the imager of the dummy device at the distal end of the applicator;
retracting the dummy device into the afterloader after the focused image has been taken;
determining an exact location of the applicator based on one or both the anatomical image and the focused image;
determining a treatment time for a radiation source for the applicator based on the location of the tumor, any organs at risk, and the exact location of the applicator.

12. The method of claim 11, wherein anatomical image data from the anatomical image includes one of: image data of the applicator, no image data of the applicator, or a digital representation of the applicator.

13. The method of claim 12, further comprising driving the radioactive source from the output channel of the afterloader to the distal end of the applicator.

14. The method of claim 13, further comprising:
starting the treatment time after the radioactive source has been placed at the distal end of the applicator; and
retracting the radioactive source after the treatment time has expired.

15. A method of operating a brachytherapy treatment system, the method comprising:
taking an anatomical image of a patient to determine shapes and locations of a tumor and any organs at risk;
sequentially driving a dummy device from a plurality of output channels of an afterloader to the distal ends of a plurality applicators, the dummy device having a high contrast to an imager;
taking a plurality of focused images with the imager of the dummy device such that a focused image is taken of the dummy device at the distal end of each applicator;
retracting the dummy device into the afterloader after each focused image has been taken;
determining the exact locations of the applicators based on one or both the anatomical image and the focused images;
determining a treatment time for a radiation source for the applicator associated with each output channel based on the location of the tumor, any organs at risk, and the exact locations of the distal ends of the applicators.

16. The method of claim 15, wherein anatomical image data from the anatomical image includes one of: image data of the applicator, no image data of the applicator, or a digital representation of the applicator.

17. The method of claim 16, further comprising driving the radioactive source from the output channel of the afterloader to the distal end of the applicator.

18. The method of claim 17, further comprising:
starting the treatment time after the radioactive source has been placed at the distal end of the applicator; and
retracting the radioactive source after the treatment time has expired.

19. The method of claim 18, further comprising:
coupling the applicator to a guide tube; and
randomly coupling the number of guide tubes to a number of output channels of the afterloader so that each guide tube is coupled to an output channel.

20. The method of claim 19 wherein the afterloader determines whether a channel identified by a channel number is coupled to an applicator.
